(19) United States
(12) Patent Application Publication
(10) Pub. No.: US 2020/0345810 A1
(43) Pub. Date: Nov. 5, 2020

(54) USE OF ALPHA-1-MICROGLOBULIN FOR PROTECTION OF BONE MARROW CELLS

(71) Applicant: GUARD THERAPEUTICS INTERNATIONAL AB, Lund (SE)

(72) Inventors: Johan Flygare, Lund (SE); Eddie Thordarson, Bjärred (SE); Bo Åkerström, Lund (SE); Magnus Göran Gram, Oxie (SE)

(21) Appl. No.: 16/760,724

(22) PCT Filed: Nov. 1, 2018
(86) PCT No.: PCT/EP2018/079926

§ 371 Date: Apr. 30, 2020

(30) Foreign Application Priority Data
Nov. 3, 2017 (DK) PA 2017 70823

(51) Int. Cl. A61K 38/17; A61P 39/00; A61P 7/00

(52) U.S. Cl. CPC A61K 38/1722; A61P 7/00; A61P 39/00

(57) ABSTRACT

The present invention relates to alpha-1-microglobulin (A1M) for use in the protection of bone marrow cells in a subject.

### FIELD OF THE INVENTION

The present invention relates to the use of alpha-1-microglobulin (A1M) for protection of bone marrow cells, especially against damage to hematopoietic stem or progenitor cells residing in the bone marrow or other hematological niches. Damage to such cells may occur in connection with exposure to radiation, chemotherapy or other genotoxic agents. The radiation may be occupational, accidental such as e.g. exposure to radiation from nuclear plants, mining industries, nuclear weapon etc. or it may be ionizing radiation associated with medical screening, diagnosis, and treatment.

### BACKGROUND OF THE INVENTION

Hematopoiesis, the process of blood cell formation, occurs during embryonic development and throughout adulthood to produce and replenish the blood system. Blood is one of the most highly regenerative tissues. In fact, over 90% of all cells in the body are hematopoietic cells and approximately one trillion (10¹²) new blood cells are produced daily. Much of our understanding of human hematopoiesis comes from studying mouse hematopoiesis and through human-mouse xenotransplantation studies. In adults hematopoiesis is mainly sustained by hematopoietic stem cells (HSC) residing in the bone marrow. HSCs are critical for lifelong blood production and HSCs are uniquely defined by their capacity to durably self-renew, or generate daughter stem cells, while still contributing to the pool of differentiating cells. HSCs sit atop a hierarchy of progenitors that become progressively restricted to several or single lineages. These progenitors yield blood precursors devoted to unilineage differentiation and production of mature blood cells, including red blood cells, megakaryocytes, myeloid cells (monocyte/macrophage and neutrophil), and lymphocytes. In children, hematopoiesis occurs in the marrow of the long bones such as the femur and tibia. In adults, it occurs mainly in the pelvis, cranium, vertebrae, and sternum. However, maturation, activation, and some proliferation of certain hematopoietic cells occurs in extramedullary hematopoietic niches such as the peripheral blood, spleen, liver, thymus, and lymph nodes.

Radiation is often used to treat malignancies such as various forms of cancer. However, it is well established that exposure to ionizing radiation causes a drastic deficit to bone marrow cell populations and even sub-lethal doses may lead to irreparable tissue damage of the hematopoietic stem and progenitor cells residing in the bone marrow and other hematopoietic niches. Bone marrow toxicity from radiation results from damage to HSC as well as bone microenvironment. Exposure to high doses of ionizing radiation or chemotherapy will lead to bone marrow failure and ultimately may lead to death. However, high doses of ionizing radiation as well as chemotherapy are often beneficial for pursuing medical treatment. Many patients with hematological disorders or cancers of the bone marrow undergo elective radiation- or chemotherapy and patients with tumors may also undergo localized radiotherapy.

Patients undergoing radiation therapy or otherwise being exposed to radiation may suffer from long-term effect on bone. Moreover, as cancer patients continue to undergo whole body or localized radiation therapy, it is important to develop means for rescuing or protecting the hematopoietic cells residing in the bone marrow and other hematologic niches.

WO 2010/006809 relates to broad antioxidative properties of A1M, and suggests using A1M in diseases involving oxidative stress such as infection and inflammation, ischemia- and reperfusion-related diseases, oxidative stress as a result of free hemoglobin, heme and iron ions, environmental and food derived factors, disorders of the skin, reproduction, and neonatal medicine.

WO 2016/135214 relates to the use of A1M in the treatment of acute and/or chronic kidney injuries and in kidney-related side effects observed in radionuclide diagnostics (RD), radionuclide therapy (RNT) and radioimmunotherapy (RIT).

Gunnarsson Rolf et al. 2016 and Lena Wester-Rosenlof et al. 2014 both relate to A1M in the treatment of preeclampsia. Lena Wester-Rosenlof et al. 2014 studies blood, placenta tissue and kidney tissue in a PE ewe model.

### DESCRIPTION OF THE INVENTION

As demonstrated in the examples herein the present inventors have found that A1M protects against damage to cells residing in the bone marrow or other hematological niches during or following ionizing radiation. It is envisaged that the protective effect of A1M on the hematopoietic cells, e.g. in bone marrow, is not limited to radiation exposure, but may be equally protective in other situations, e.g. following exposure to chemotherapeutics; chemicals; viruses or other toxins, negatively affecting the cells in the bone marrow and other hematological niches like e.g. the spleen. In the present examples, focus has been on the negative effects on bone marrow following ionizing radiation.

The new findings are totally unexpected given the knowledge the inventors have today. Previous reports show that A1M protects the kidneys as a result of A1M's biodistribution to the kidneys, but do not support or suggest that A1M is localized to bone marrow cells and hence it could not have been foreseen that A1M can protect hematopoietic cells residing in the bone marrow.

More specifically, the invention relates to:

Alpha-1-microglobulin (A1M) for use in the protection bone marrow cells such as hematopoietic stem and/or progenitor cells residing in the bone marrow or other hematological niches such as peripheral blood, spleen, liver, thymus, and lymph nodes. Some proliferation of HSCs occurs in the spleen, liver, thymus and lymph nodes (hematological niches);

A1M for use in the protection of bone marrow cells such as hematopoietic stem and/or progenitor cells residing in the bone marrow or other hematological niches, wherein the damage is caused by exposure to ionizing radiation, chemotherapeutics, viruses, or other toxic substances. Radiation may be in connection with medical treatment or it may be accidental radiation associated with nuclear plants, exposure to nuclear weapons etc;

A1M for use in the protection of bone marrow cells such as protection of damage to hematopoietic stem and/or progenitor cells residing in the bone marrow or other hematological niches caused by exposure to ionizing radiation; A1M may be administered before, during and/or after exposure to the radiation;

A1M for use in the protection of bone marrow cells such as protection of damage to hematopoietic stem and/or progenitor cells residing in the bone marrow or other hematological niches following exposure to ionizing radiation, wherein A1M is used as a co-treatment to the radiation;

A1M and a compound labeled with radionuclide for use in the co-treatment of malignancies requiring radiation therapy, wherein A1M is used to avoid or reduce damage to bone marrow cells such as hematopoietic stem and/or progenitor cells residing in the bone marrow or other hematological niches caused by ionizing radiation;

A1M and a chemotherapeutic substance for use in the co-treatment of malignancies requiring chemotherapy, wherein A1M is used to avoid or reduce damage to bone marrow cells such as hematopoietic stem and/or progenitor cells residing in the bone marrow or other hematological niches caused by the chemotherapy;

A1M for use in the treatment of damage to bone marrow cell such as hematopoietic stem and/or progenitor cells residing in the bone marrow or other hematological niches;

A1M for use in the treatment of bone marrow injuries. The injuries are damage to hematopoietic stem and/or progenitor cells residing in the bone marrow or other hematological niches;

A1M for use in reducing the unwanted biological effect on bone marrow cells from ionizing radiation e.g. during radionuclide diagnostics (nuclear medicine imaging) in single or multiple imaging sessions. A1M is used to achieve the ALARA principle (As Low As Reasonably Achievable) and reduce the unwanted effect of ionizing radiation to the patient. The unwanted effect being negative effects on hematological cells residing in the bone marrow or other hematological niches.

In any event, it is contemplated that A1M has a protective effect on bone marrow cells such as hematopoietic stem and/or progenitor cells residing in the bone marrow or other hematological niches.

In the experimental section herein PRRT (peptide receptor radionuclide therapy) has been used together with A1M to demonstrate A1M's protective effect on the damaging effect on hematopoietic stem and/or progenitor cells residing in the bone marrow or other hematological niches caused by PRRT. It is envisaged that A1M will have similar protective effects on other types of ionizing radiation that cause negative effects on the hematopoietic cells residing in the bone marrow or other kinds of exposure that cause negative effects, e.g. chemotherapy or other genotoxic agents, on the hematopoietic stem and/or progenitor cells residing in the bone marrow or other hematological niches. Thus, the invention is not limited to a specific peptide receptor radionuclide (PRRN) such as those mentioned below, but any source of ionizing radiation, including external beam radiation, is within the scope of the present invention. Thus, included in the present context is also radionuclide diagnostics (RD), radionuclide therapy (RNT) and radioimmunotherapy (RIT) as well as any molecule labelled with any suitable radionuclide capable of emitting ionizing radiation is intended to be within the scope of the present invention, such as the radionuclide-labelled small molecules Affibody molecules, Dia-bodies, Fab, Fv, scFv-fragments and other immunoconjugates or other receptor ligands. The radiation may be for therapy or medical treatment, but the radiation may also be accidentally such as occupational exposure. In the present context, radiation for medical use is preferred. In the event that the radiation is as an occupational, accidental or medical exposure, A1M may be administered either before, during or after the exposure. As mentioned herein before, included in the scope of the present invention is also the use of A1M to protect against damage to hematopoietic stem and/or progenitor cells residing in the bone marrow or other hematological niches caused by other types of exposure than ionizing radiation, e.g. chemotherapy or genotoxic agents.

PRRT is a form of molecular targeted therapy, which is performed by use of a small peptide coupled to a radionuclide emitting radiation. In the examples described by the inventors herein, the small peptide is a somatostatin analogue. However, these peptides can also include octreotide, lanreotide, Tyra-octreotide (TOC), Tyr³-octrotate (TATE) and the DOTA⁺-chelates DOTADOC, DODATATE and DOTA-lanreotide. Other somatostatin analogues include SOM230 (pasireotide), dopastatin and octreotide LAR. In the case of PRRT, the somatostatin analogues are labelled with radionuclides emitting medium and/or high energy beta particles such as Yttrium-90 (⁹⁰Y) or Lutetium-177 (¹⁷⁷Lu) and administered to the patient intravenously (i.v.). However, the radionuclide could be of another type, such as Indium-111 (¹¹¹In). The somatostatin related therapy is conducted on patients having somatostatin receptor positive tumors. Many, but not all, forms for neuroendocrine tumors (NETs) express one or more somatostatin receptor subtype. After administration of a PRRN it binds to the somatostatin receptor localized on the tumor and the PRRN is retained in the tumor. The decay of the radionuclide emitting ionizing radiation deposits energy in the tissues resulting in a high absorbed dose.

In the present context the invention is not limited to a specific PRRN such as those mentioned above. The invention also includes any molecule labelled with any suitable radionuclide capable of emitting ionizing radiation, such as the radionuclide-labelled small molecules Affibody molecules, Diabodies, Fab, Fv, scFv-fragments and other immunoconjugates or other receptor ligands. Moreover, as mentioned herein before, the scope of the invention also includes other types of ionizing radiation and other causes of damage to hematopoietic stem and/or progenitor cells residing in the bone marrow or other hematological niches.

In the present context, the terms “bone marrow failure”, “bone marrow damage”, “impaired hematopoietic stem and/or progenitor cell function” or “negative effects on bone marrow” are used interchangeably and are defined as any acute or chronic impairment of normal hematopoietic stem and/or progenitor cell function.

NETs are examples of tumors, where radiation is applied. NETs are a large group of slowly growing neoplasms derived from the neuroendocrine system, characterized by their overexpression of hormone receptors. Most NETs originate in the gastroenteropancreatic (GEP) and bronchopulmonary tract, and their incidence and prevalence have been steadily increasing over the past three decades. The surveillance, epidemiology, and end results (SEER) program of the National Cancer Institute in the United States reported an increase from 1.09 cases per 100,000 in 1973 to 5.25 cases per 100,000 in 2004 (n=35 825), probably as result of trends in imaging and improvement in diagnosis. Women and men are affected equally, and the prevalence of NETs is reported to be 35 per 100 000. NET tumors are classified based on the proportion of proliferating cells in the tumor as determined by the proliferation marker Ki-67. NETs with a Ki-67-index of 0-2% are classified as Grade 1 (G1), those with 3-20% as Grade 2 (G2), and NETs with >20% as Grade 3 (G3). The median overall survival time is 75 months, but the prognosis varies according to the origin, stage and grade of disease.

NETs are biologically and clinically heterogeneous and the rates and locations of metastatic spread, patterns of hormonal secretion and survival outcomes vary greatly between tumors of different primary sites. For example, NETs of the small intestine have a relatively high malignant potential but tend to progress indolently while gastric, or rectal NETs usually display a low malignant potential but behave aggressively in the advanced setting. Metastatic midgut NETs often secrete serotonin and other vasoactive substances, giving rise to the typical carcinoid syndrome, primarily characterized by flushing, diarrhea, and right-sided valvular heart disease.

Surgery is the only curative treatment for localized NETs. However, more than 40% of patients have metastatic disease at diagnosis, thus requiring systemic treatments. Over the last few years, the therapeutic landscape of advanced NETs has undergone a remarkable expansion, and targeted agents including somatostatin analogs (SSAs), everolimus, and sunitinib have demonstrated safety and efficacy. PRRT, a form of systemic radiotherapy that allows targeted delivery of radionuclides to tumor cells expressing high levels of somatostatin receptors (SSTRs), has shown significant promise for the treatment of advanced, low- to intermediate-grade NETs. The effects of PRRT are mediated through interaction with five SSTRs (SSTR1-5). NETs are characterized by the high-density expression of SSTRs. Upon receptor binding, radiolabeled SSAs are internalized and the breakdown products of the radiolabeled peptides are stored in lysosomes, thus enabling delivery and retention of radioactivity into the tumor cell interior. Radiolabeled SSAs consist of a radionuclide isotope, a carrier molecule (octreotide derivative), and a chelator that binds them and stabilizes the complex. Commonly used chelators include DOTA (tetraazacyclododecane-tetra-acetic acid) and DTPA (diethylenetriamine penta-acetic acid), while octreotide and octreotate, analogues with enhanced affinity to SSTR2, are generally used as carriers. Three radionuclides (¹¹¹In, ⁹⁰Y, and ¹⁷⁷Lu) have been conjugated to SSAs, and their different physical characteristics confer specific benefits in radiation delivery. Such SSAs include octreotide, lanreotide, Tyra-octreotide (TOC), Tyr³-octrotate (TATE) and the DOTA⁺-chelates DOTADOC, DODATATE and DOTA-lanreotide. Other somatostatin analogues include SOM230 (pasireotide), dopastatin and octreotide LAR. The somatostatin analogues are labelled with radionuclides emitting medium and/or high energy beta particles such as ⁹⁰Y¹⁷⁷Lu and administered to the patient i.v. In clinical studies, including the randomized, phase III NETTER-1 trial, ¹⁷⁷Lu is most commonly used.

PRRT is the only treatment option for NETs with a clear predictive biomarker: SSTR expression. Increased response rates have been demonstrated in patients with higher degree of radiotracer uptake on SSTR scintigraphy (octreoscan), and an overall response rate (ORR) of ˜60% has been reported for patients with grade 4 uptake by Krenning score (tumor uptake greater than that of the spleen or kidneys). The activity of PRRT is also influenced by the site of the primary tumor and the tumor load. The intended cumulative dose of radiolabeled SSAs is fractionated in sequential cycles (usually four to five), delivered systemically every 6-9 weeks. Importantly, treatment can only be repeated to a limited extent, because of the limitations imposed by bone marrow and kidney irradiation. In terms of radiotoxicity, the side effects associated with PRRT can be categorized as acute and delayed. Acute effects include nausea, vomiting and abdominal pain. These reactions are often normalized after the end of therapy. Also regarded as acute is bone marrow and hematological effects that can be observed after treatment. Consequently, the successful development of a drug to protect normal tissue from radiation-induced damage, particularly bone marrow, would enable a more effective cancer therapy and improved patient health.

### Alpha-1-Microglobulin—a General Background

A1M is synthesized in the liver at a high rate, secreted into the blood stream and transported across the vessel walls to the extravascular compartment of all organs. The protein is also synthesized in other tissues (blood cells, brain, kidney, skin) but at a lower rate. Due to the small size, free A1M is rapidly filtered from blood in the kidneys.

A1M is a member of the lipocalin superfamily, a group of proteins from animals, plants and bacteria with a conserved three-dimensional structure but very diverse functions. Each lipocalin consists of a 160-190-amino acid chain that is folded into a β-barrel pocket with a hydrophobic interior. At least twelve human lipocalin genes are known. A1M is a 26 kDa plasma and tissue protein that so far has been identified in mammals, birds, fish and frogs. The three-dimensional structure of A1M, as determined by X-ray crystallography, is shown in FIG. 1. A1M is synthesized in the liver at a high rate, secreted into the blood stream and rapidly (T½=2-3 min) transported across the vessel walls to the extravascular compartment of all organs. A1M is found both in a free, monomeric form and as covalent complexes with larger molecules (IgA, albumin and prothrombin) in blood and interstitial tissues. Due to the small size, free A1M is rapidly filtered from blood in the kidneys. The major portion is then reabsorbed, but significant amounts are excreted to the urine.

### Sequence and Structural Properties of A1M

The full sequence of human A1M is known. The protein consists of a polypeptide with 183 amino acid residues. Many additional A1M cDNAs and/or proteins have been detected, isolated and/or sequenced from other mammals, birds, amphibians, and fish. The length of the peptide chain of A1M differs slightly among species, due mainly to variations in the C-terminus. Alignment comparisons of the different deduced amino acid sequences show that the percentage of identity varies from approximately 75-80% between rodents or ferungulates and man, down to approximately 45% between fish and mammals. A free cysteine side-chain at position 34 is conserved. This group has been shown to be involved in redox reactions (see below), in complex formation with other plasma proteins and in binding to a yellow-brown chromophore. The three-dimensional structure of A1M shows that C34 is solvent exposed and located near the opening of the lipocalin pocket (see FIG. 1).

In the present context the term “alpha-1-microglobulin” or the corresponding abbreviation “A1M” intends to cover alpha-1-microglobulin as identified in SEQ ID NO: 1 (wild type human A1M) as well as SEQ ID NO: 2 (human recombinant A1M) as well as any homologues, fragments or variants thereof having similar therapeutic activities. Thus, A1M as used herein is intended to mean a protein having at least 80% sequence identity with SEQ ID NO:1 or SEQ ID NO:2, or a fragment thereof. It is preferred that A1M as used herein has at least 90% sequence identity with SEQ ID NO:1 or SEQ ID NO:2. It is even more preferred that A1M as used herein has at least 95% such as 99% or 100% sequence identity with SEQ ID NO:1 or SEQ ID NO:2. In a preferred aspect, the A1M is in accordance with SEQ ID NO: 1 or 2 as identified herein. In the sequence listing is given the sequence listing of the amino acid sequence of human A1M and human recombinant A1M (SEQ ID NOs 1 and 2, respectively) and the corresponding nucleotide sequences (SEQ ID NOs 3 and 4, respectively). However, homologues, variants and fragments of A1M having the important parts of the proteins as identified in the following are also comprised in the term A1M as used herein.

### Details on Alignment/Identity

Positions of amino acid residues herein refer to the positions in human A1M as it is found in human blood plasma (SEQ ID NO:1). When referring to amino acid residues in recombinant A1M, which harbours a methionine or N-formyl methionine residue N-terminally linked to the glycine residue that is the initial residue in A1M (SEQ ID NO: 2), or in mutated human A1M or A1M from other species a person skilled in the art will understand how to identify residues corresponding to residues in human A1M (SEQ ID NO:1) even when positions are shifted due to e.g. deletions or insertions.

When recombinant proteins are produced they most often start with an initial Met residue, which may be removed using e.g. a methionine aminopeptidase or another enzyme with a similar activity. The A1M variants presented here may be with or without an initial Met residue.

### Homologues of A1M

As mentioned above homologues of A1M can also be used in accordance with the description herein. In theory A1M from all species can be used for the purposes described herein including the most primitive found so far, which is from fish (plaice).

A1M is also available in isolated form from human, orangutan, squirrel monkey, rat, naked mole rat, mouse, rabbit, guinea pig, cow, frog, chicken, walrus, manatee and plaice.

Considering homologues, variants and fragments of A1M, the following has been identified as important parts of the protein:

Y22 (Tyrosine, pos 22, basepairs 64-66)
C34 (Cystein, position 34, basepairs 100-102)
K69 (Lysine, pos 69, basepairs 205-207)
K92 (Lysine, pos 92, basepairs 274-276)
K118 (Lysine, pos 118, basepairs 352-354)
K130 (Lysine, pos 130, basepairs 388-390)
Y132 (Tyrosine, pos 132, basepairs 394-396)
L180 (Leucine, pos 180, basepairs 538-540)
I181 (Isoleucine, pos 181, basepairs 541-543)
P182 (Proline, pos 182, basepairs 544-546)
R183 (Arginine, pos 183, basepairs 547-549)
(Numbering of amino acids and nucleotides throughout the document refers to SEQ ID 1 and 3; if other A1M from other species, A1M analogs or recombinant sequences thereof are employed, a person skilled in the art will know how to identify the amino acids of the active site(s) or site(s) responsible for the enzymatic activity.)

Thus, in those cases, where A1M e.g. has 80% (or 90% or 95%) sequence identity with one of SEQ ID NO: 1 or 2, it is preferred that the amino acids mentioned above are present at the appropriate places in the molecule.

### A1M Mutations

As mentioned above, A1M may be used as the wild type or a human recombinant A1M, or homologues hereof. Moreover, the following point mutations in the A1M gene are of particular interest in the present invention:
Point mutation in A1M-gene leading to expression of His instead of Arg at position 66 (R66H),
Point mutations in A1M-gene leading to expression of Asp instead of Asn at positions 17 and 96 (N17,96D),
Point mutation in A1M-gene leading to expression of Met instead of Lys at position 41 (M41K).

These point mutations are found to improve stability with maintained function of A1M and can be useful for protecting bone marrow cells, hematopoietic stem cells, and/or progenitor cells.

Mutations (M41K+R66H), (M41K+N17,96D), (R66H+N17,96D), and/or (M41K+R66H+N17,96D) have showed increased solubility and/or stability with maintained function. Mutation (R66H+N17,96D) showed overall good performance.

### Other A1M Variants

Furthermore, truncation of the C-terminal of A1M, so that the C-terminal tetrapeptide sequence LIPR does not form part of the protein, seems to impart improved heme binding and degradation.

In addition, the influence of N-terminal, charged and hydrophilic extensions can be modified in the A1M-variants. The N-terminal extensions can be modified by 1) a tag for purification (e.g. His-tag), 2) a linker to separate the tag from the core of the A1M protein, 3) several (1-5) charged amino acid side-groups conferring hydrophilic properties to the protein in order to gain maximal stability and solubility in water-solutions, without compromising the physiological functions of A1M.

A1M with or without the following initial sequences (peptides) can be used:
M8H5GIEGR: peptide with the amino acid sequence MHHHHHHHHGGGGGIEGR or another relevant tag (HHHHHHHH) and linker (GGGGGIEGR)
M8H4DK: peptide with the amino acid sequence MHHHHHHHHDDDDK or another relevant tag (HHHHHHHH) or linker (DDDDK)
M6H4DK: peptide with the amino acid sequence MHHHHHHDDDDK or another relevant tag (HHHHHH) or linker (DDDDK)
M8H: peptide with the amino acid sequence MHHHHHHHH

Based on these observations, it is contemplated that variation of an A1M protein along the lines indicated above will provide proteins with A1M functionality, but with improved characteristics regarding stability and/or solubility.

Thus, the present invention also relates to all possible combinations of A1M containing modifications to the N-terminal, e.g. His-tag, truncated C-terminally, i.e. without LIPR, and any combination of the point mutations M41K, R66H, N17,96D.

In the following a listing of the sequences are given. The invention encompass all possible variations e.g. such as those illustrated herein.

SEQ ID NO: 1: wt hA1M (protein)
SEQ ID NO: 2: rhA1M (i.e. Met-A1M) (protein)
SEQ ID NO: 3: wt hA1M (nucleotide sequence)
SEQ ID NO: 4: rhA1M (i.e. Met-A1M) (nucleotide sequence)
SEQ ID NO: 5: Preferred mutation without extension—N17,96D, R66H
SEQ ID NO: 6: No extension, M41K
SEQ ID NO: 7: Preferred mutation with 6 His, N17,96D, R66H

### SEQ ID NO: 8: 6His, M41K

SEQ ID NO: 9: Preferred mutation with 8 His extension, N17,96D, R66H

### SEQ ID NO: 10:8 His, M41K

SEQ ID NO: 11: Extension+wt hA1M
SEQ ID NO: 12: Preferred mutation without extension—N17,96D, R66; C-terminally truncated
SEQ ID NO: 13: No extension, M41K; C-terminally truncated
SEQ ID NO: 14: Preferred mutation with 6 His, N17,96D, R66H; C-terminally truncated
SEQ ID NO: 15: 6His, M41K; C-terminally truncated
SEQ ID NO: 16: Preferred mutation with 8 His extension, N17,96D, R66H; C-terminally truncated
SEQ ID NO: 17:8 His, M41K; C-terminally truncated

It is contemplated that the N-terminal tag (6 or 8 His) may be replaced by any other suitable tag for preparative or isolation purposes. Moreover, the linker between the N-terminal tag and the core A1M molecule may also be varied in number and individually selected from Asp, Glu, Lys or Arg.

### Further to A1M

Human A1M is substituted with oligosaccharides in three positions, two sialylated complex-type, probably diantennary carbohydrated linked to N17 and N96 and one more simple oligosaccharide linked to T5. The carbohydrate content of A1M proteins from different species varies greatly, though, ranging from no glycosylation at all in Xenopus leavis over a spectrum of different glycosylation patterns. However, one glycosylation site, corresponding to N96 in man, is conserved in mammals, suggesting that this specific carbohydrate may be functionally important.

A1M is yellow-brown-colored when purified from plasma or urine. The color is caused by heterogeneous compounds covalently bound to various amino acid side groups mainly located at the entrance to the pocket. These modifications represent the oxidized degradation products of organic oxidants covalently trapped by A1M in vivo, for example heme, kynurenine and tyrosyl radicals.

A1M is also charge- and size-heterogeneous and more highly brown-colored A1M-molecules are more negatively charged. The probable explanation for the heterogeneity is that different side-groups are modified to a varying degree with different radicals, and that the modifications alter the net charge of the protein. Covalently linked colored substances have been localized to C34, and K92, K118 and K130, the latter with molecular masses between 100 and 300 Da. The tryptophan metabolite kynurenine was found covalently attached to lysyl residues in A1M from urine of hemodialysis patients and appears to be the source of the brown color of the protein in this case [6]. Oxidized fragments of the synthetic radical ABTS (2,2′-azino-di-(3-ethylbenzothiazoline)-6-sulfonic acid) was bound to the side-chains of Y22 and Y132.

C34 is the reactive center of A1M. It becomes very electronegative, meaning that it has a high potential to give away electrons, by the proximity of the positively charged side-chains of K69, K92, K118 and K130, which induce a deprotonization of the C34 thiol group which is a prerequisite of oxidation of the sulphur atom. Preliminary data shows that C34 is one of the most electronegative groups known.

Theoretically, the amino acids that characterize the properties of A1M (C34, Y22, K92, K118, K130, Y132, L180, I181, P182, R183), which will be described in more detail below, can be arranged in a similar three-dimensional configuration on another framework, for instance a protein with the same global folding (another lipocalin) or a completely artificial organic or inorganic molecule such as a plastic polymer, a nanoparticle or metal polymer.

The three-dimensional arrangement of some of these amino acids (blue ovals, the lysines are depicted by a “+”), the A1M-framework (barrel), the electron-flow and the radical-trapping, are illustrated in FIG. 1.

Accordingly, homologues, fragments or variants comprising a structure including the reactive center and its surroundings as depicted above, are preferred.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids the hydrophilicity values of which are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln₁ His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Lle, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

In the present context, the homology between two amino acid sequences or between two nucleic acid sequences is described by the parameter “identity”. Alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6.

Multiple alignments of protein sequences may be made using “ClustalW”. Multiple alignments of DNA sequences may be done using the protein alignment as a template, replacing the amino acids with the corresponding codon from the DNA sequence.

Alternatively different software can be used for aligning amino acid sequences and DNA sequences. The alignment of two amino acid sequences is e.g. determined by using the Needle program from the EMBOSS package (http://emboss.org) version 2.8.0. The Needle program implements the global alignment algorithm described in. The substitution matrix used is BLOSUM62, gap opening penalty is 10, and gap extension penalty is 0.5.

The degree of identity between an amino acid sequence; e.g. SEQ ID NO: 1 and a different amino acid sequence (e.g. SEQ ID NO: 2) is calculated as the number of exact matches in an alignment of the two sequences, divided by the length of the “SEQ ID NO: 1” or the length of the “SEQ ID NO: 2”, whichever is the shortest. The result is expressed in percent identity.

An exact match occurs when the two sequences have identical amino acid residues in the same positions of the overlap.

If relevant, the degree of identity between two nucleotide sequences can be determined by the Wilbur-Lipman method using the LASER-GENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

The percentage of identity of an amino acid sequence of a polypeptide with, or to, amino acids of SEQ ID NO: 1 may be determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention is calculated in an analogous way.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylpróline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. Naturally occurring amino acid residues can be converted to non-naturally occurring species by in vitro chemical modification. Chemical modification can be combined with site-directed mutagenesis to further expand the range of substitutions. Alternative chemical structures providing a 3-dimensional structure sufficient to support the properties of A1M may be provided by other technologies e.g. artificial scaffolds, amino-acid substitutions and the like. Furthermore, structures mimicking the active sites of A1M as listed above are contemplated as having the same function as A1M.

### Pharmaceutical Compositions and Dosage

The present invention also relates to

i) the use of a pharmaceutical composition comprising A1M for protection of bone marrow cells such as hematopoietic stem and/or progenitor cells residing in the bone marrow or other hematological niches;
ii) the use of a pharmaceutical composition comprising A1M for protection of bone marrow cells such as hematopoietic stem and/or progenitor cells residing in the bone marrow or other hematological niches, wherein the damage is caused by ionizing radiation;
iii) the use of a pharmaceutical composition comprising A1M for protection bone marrow cells such as hematopoietic stem and/or progenitor cells residing in the bone marrow or other hematological niches, wherein the damage is caused by chemotherapeutics;
iv) the use of a pharmaceutical composition comprising A1M for protection of bone marrow cells such as hematopoietic stem and/or progenitor cells residing in the bone marrow or other hematological niches, wherein the damage is caused by a toxic substance;
v) the use of a pharmaceutical composition comprising A1M for preventing the damages to the bone marrow cells mentioned above, wherein the composition comprising A1M is administered before any treatment with ionizing radiation or with chemotherapeutics;
vi) a kit comprising:
a) means for radiation therapy, and
b) a pharmaceutical composition comprising A1M, for protection of bone marrow cells such as hematopoietic stem and/or progenitor cells residing in the bone marrow or other hematological niches caused by ionizing radiation;
vii) a kit as mentioned above, wherein the means for radiation therapy is a pharmaceutical composition comprising a PRRN;
for protection of bone marrow cells such as hematopoietic stem and/or progenitor cells residing in the bone marrow or other hematological niches caused by ionizing radiation;
viii) a kit comprising
a) means for chemotherapy, and
b) a pharmaceutical composition comprising A1M, for protection of bone marrow cells such as hematopoietic stem and/or progenitor cells residing in the bone marrow or other hematological niches caused by chemotherapy

A kit may be in the form of one package containing the above-mentioned two compositions.

Pharmaceutical compositions comprising means for radiation therapy such as PRRN, or comprising means for chemotherapy are typically a composition already on the market.

The pharmaceutical composition comprising A1M (or an analogue, fragment or variant thereof as defined herein) is intended for any suitable administration route including parenteral administration such as i.v. or subcutaneous administration. Accordingly, A1M can be formulated in a liquid, e.g. in a solution, a dispersion, an emulsion, a suspension etc. A suitable vehicle for i.v. administration may be composed of 10 mM Tris-HCl, pH 8.0 and 0.125 M NaCl. Another suitable vehicle for i.v. administration may be composed of 10 mM Na-phosphate, pH 7.4, 0.15 M NaCl and 2 mg/mL histidine.

For parenteral use suitable solvents include water, vegetable oils, propylene glycol and organic solvents generally approved for such purposes. In general, a person skilled in the art can find guidance in “Remington's Pharmaceutical Science” edited by Gennaro et al. (Mack Publishing Company), in “Handbook of Pharmaceutical Excipients” edited by Rowe et al. (PhP Press) and in official Monographs (e.g. Ph.Eur. or USP) relating to relevant excipients for specific formulation types and to methods for preparing a specific formulation.

In those cases, where A1M is used in conjunction to radiation exposure, radiation therapy, chemotherapy, or exposure to other genotoxic agents, A1M will be administrated in one or several doses in connection to the exposure or therapy. Preferably, each dose will be administrated e.g. i.v. or subcutaneous or through any other available route either as a single or multiple dose. The first dose will be administrated at the same time as the exposure or therapy, before or after exposure or therapy. Additional A1M-doses can be added, but may not be necessary, after exposure or therapy. Each dose contains an amount of A1M which is related to the bodyweight of the patient: 0.1-100 mg A1M/kg of the patient. In the study described in the examples herein a dose of 20 mg/kg (administered subcutaneously in a mouse model) is employed.

In those cases where the hematopoietic stem cells and/or progenitor cells in the bone marrow are already injured and no radiation therapy or chemotherapy is required, A1M can be administered as described above or in multiple doses.

The effect of the treatment with A1M may be followed by for instance, but is not limited to, measurement of the percentage of reticulocytes in peripheral blood compared with percentage of reticulocytes in peripheral blood from the same patient, but where the blood sample is drawn before treatment, where an increase in percentage denotes a positive effect on the bone marrow. Alternatively, the comparison can be with a control sample from healthy volunteers.

### Sequence Listing Free Text

### SEQ ID NO: 1

<223> Wild type human A1M, no mutations

### SEQ ID NO: 2

<223> rhA1M, i.e. N-terminal Met

### SEQ ID NO: 3

<223> Wild type human A1M, no mutations (nucleotide sequence)

### SEQ ID NO: 4

<223> rhA1M, i.e. N-terminal Met (nucleotide sequence)

### SEQ ID NO: 5

<223> hA1M, no tag, N-terminal Met, N17,96D; R66H

### SEQ ID NO: 6

<223> hA1M, no tag, N-terminal Met, M41K

### SEQ ID NO: 7

### <223>6His, N17,96D; R66H

### SEQ ID NO: 8

<223> hA1M, 6His, M41K

### SEQ ID NO: 9

### <223>8His, N17,96D; R66H

### SEQ ID NO: 10

<223> hA1M, 8His, M41K

### SEQ ID NO: 11

<223> hA1M, 8His, no mut

### SEQ ID NO: 12

<223> hA1M, no tag, N-terminal Met, N17,96D; R66H; truncated

### SEQ ID NO: 13

<223> hA1M, no tag, N-terminal Met, M41K, truncated

### SEQ ID NO: 14

<223>6 His, N17, 96D, R66H, truncated

### SEQ ID NO: 15

<223> hA1M, 6 His, M41K, truncated

### SEQ ID NO: 16

<223>8 His, N17, 96D, R66H, truncated

### SEQ ID NO: 17

<223> hA1M, 8 His, M41K, truncated

### LEGENDS TO FIGURES

FIG. 1: Three dimensional structure of A1M with high-lighted C34 residue and marked N- and C-termini.

FIG. 2: A1M confers protection to reticulocytes within the bone marrow and peripheral blood cells following exposure to ¹⁷⁷Lu-DOTATATE.

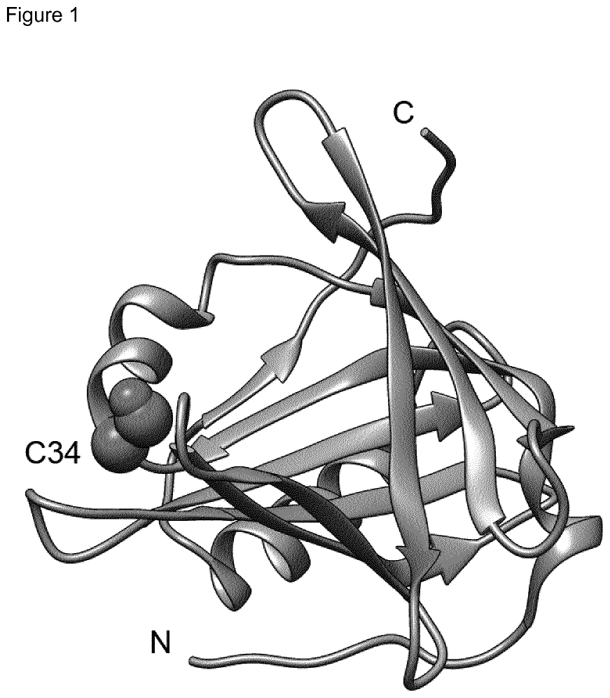

A. Single cell suspension from bone marrow were obtained by crushing femur in PBS containing 2% FCS and passing them though a 70 um cell strainer to obtain single cell suspension. Cells were blocked by incubation with mouse Fc receptor binding inhibitor and then stained with monoclonal antibodies against Ter119, CD44, CD71 and CD45. To exclude dead cells DAPI was used.

B. Peripheral blood was collected from vena saphena and reticulocytes were determined using LSR Fortessa or Canto II flow cytometry using Retic-Count.

Data is presented as mean±Std and individual data points. Differences in groups were analyzed using one-way ANOVA with post hoc Tukey.

FIG. 3: A1M confers protection of the proerythroblasts following ¹⁷⁷Lu-DOTATATE. A. Single cell suspension from bone marrow were obtained by crushing femur in PBS containing 2% FCS and passing them though a 70 um cell strainer to obtain single cell suspension. Cells were blocked by incubation with mouse Fc receptor binding inhibitor and then stained with monoclonal antibodies against Ter119, CD44, CD71 and CD45. To exclude dead cells DAPI was used.

Data is presented as mean±Std. Differences in groups were analyzed using one-way ANOVA with post hoc Tukey.

FIG. 4: A1M treatment improves expansion of erythroid cells from a murine DBA model and patients. CD117 (Kit)+ bone marrow cells were treated with Doxycycline to induce Rps19 deficiency. Twenty four hours after Doxycycline administration, cells were treated with drugs interfering with iron or heme availability. The ATP measuring platform CellTiter Glo was used to monitor viable cells after 5 days of expansion. In A) a schematic picture of the drug screen is shown. B) Proliferation of bone marrow cells (as described in A) from Rps19 inducible mice treated with respective drug compounds interfering with iron or heme availability is presented. Shown is also a schematic picture of drug activity where A1M is shown to have intracellular effects by reducing unbound heme in erythroblast. The cells were cultured in erythroid promoting media for 5 days. Kruskal-Wallis non parametric test with Dunn's multiple comparisons test was used for statistical analysis, and genotypes were compared to respective control, separately. p-values: *≤005, **≤001, ***≤0001, ****≤00001. C) Concentration of free unbound intracellular heme in Kit+ bone marrow cells from the Rps19 deficient mouse expanded in erythroid culture with A1M or vehicle for 72 hours. Mann Whitney non parametric analysis was employed for statistical analysis within each genotype. D) Expansion of CD34+ erythroid precursors from peripheral blood of DBA patients with mutations in RPS19, RPL35a and RPS26, or healthy controls treated with A1M or vehicle in erythroid promoting media for 7 days. A1M-treated or vehicle treated values from each donor is presented pairwise.

### EXPERIMENTAL

### Example 1—A1M Protects Against Radiation-Induced Damage to the Bone Marrow and Peripheral Blood Cells

In this study, we show that human recombinant A1M (A1M) confers protection against radiation-induced damage to the bone marrow and peripheral blood cells following ¹⁷⁷Lu-DOTATATE (150 MBq) exposure in BALB/c mice.

### Methods

### Recombinant Human A1M

Recombinant human A1M (A1M, variant RMC-035 corresponding to A1M (R66H+N17,96D)) were supplied by A1M Pharma AB (Lund, Sweden).

### Radiopharmaceuticals

Conjugation of radiopharmaceutical precursor's lutetium (¹⁷⁷Lu)-chloride (LuMark, IDB, Holland) and DOTA-(Tyr³)-Octreotate (ANMI, Belgium), denoted ¹⁷⁷Lu-DOTATATE, were performed at Lund University Hospital (Lund, Sweden). Quality control of the ¹⁷⁷Lu-DOTATATE conjugate was performed at Lund University Radionuclide Centre (Lund, Sweden).

### Animal Studies

Female BALB/c mice (Taconic, Denmark) at the age of 12 weeks were used in this study. Two groups (n=5-10) received a subcutaneous administration of either A1M (20 mg/kg) or vehicle buffer (10 mM Na-phosphate pH 7.4+0.15 M NaCl+12 mM histidine) followed by an intravenous (i.v.) injection, 30 minutes later, of ¹⁷⁷Lu-DOTATATE (150 MBq). A control group (n=5-10) received a subcutaneous administration of vehicle, followed by, 30 minutes after, an i.v. injections of NaCl. Animals were sacrificed 4 days post-injections.

After 4 days (post ¹⁷⁷Lu-DOTATATE administration), blood, for peripheral blood cell and reticulocyte count, was sampled from vena saphena, on non-anesthetized animals, in EDTA pre-coated vials (Microvette CB 300 K2E, Sarstedt, Nümbrecht, Germany) and placed on a rocking mixer in room temperature followed by analysis as described below. Thereafter the animals were anaesthetized using isoflurane, sacrificed by cervical dislocation and femur (left and right) sampled and placed in PBS, pH 7.4 in a 24 well plate standing on wet ice.

All animal experiments were conducted in compliance with the national legislation on laboratory animals' protection and with the approval of the Ethics Committee for Animal Research (Lund, Sweden).

### Bone Marrow Flow Cytometry Analysis:

Bone marrow cells were isolated by crushing femur in PBS containing 2% FCS (GIBCO, Waltham, Mass., USA) and passing them though a 70 um cell strainer to obtain single cell suspension. Single-cell suspensions were blocked by incubation with mouse Fc receptor binding inhibitor (eBioscience, Waltham, Mass., USA) and then stained with the following specific mouse monoclonal antibodies (mAb) purchased from BD Biosciences (Stockholm, Sweden), Ter119, CD44, CD71 and CD45. To exclude dead cells 4,6-Diamidine-2′-phenylindole dihydrochloride (DAPI) was used. All experiments were performed on LSRFortessa (BD Biosciences) flow cytometry and analyzed with FlowJo software.

Bone marrow cellularity was counted from the single cell suspension using hematology analyzer SYSMEX KX-21 N.

### Blood Analysis

Following collection of peripheral blood SYSMEX KX-21N hematology analyzer was used for determining Blood parameters. Reticulocyte count was determined using LSR Fortessa or Canto II flow cytometry using Retic-Count (BD Biosciences).

### Statistical Analysis

Results were evaluated by comparisons of all experimental groups using analysis of variance (ANOVA). All statistical calculations were made in GraphPad Prism (GraphPad Prism 7.0; GraphPad Software; GraphPad, Bethesda, Md., USA).

### Results

Analysis of hematopoietic cells in bone marrow and peripheral blood

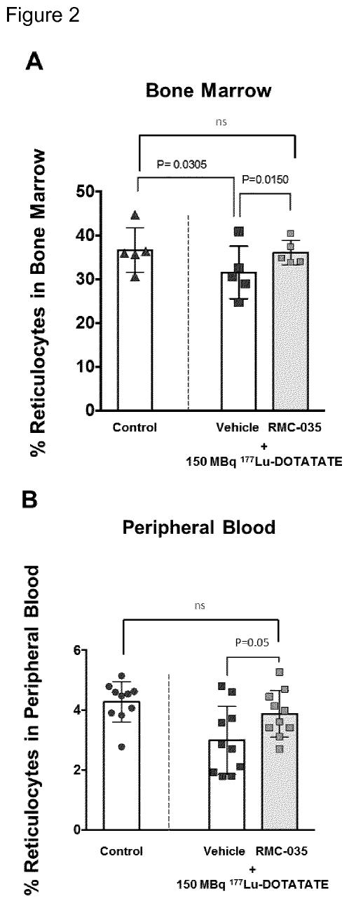

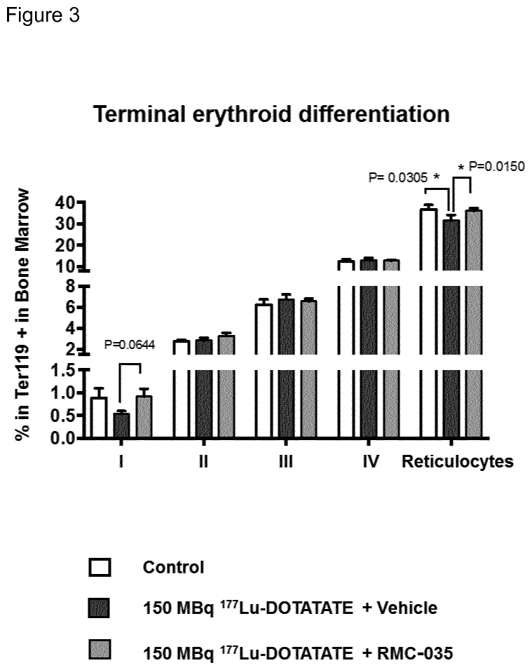

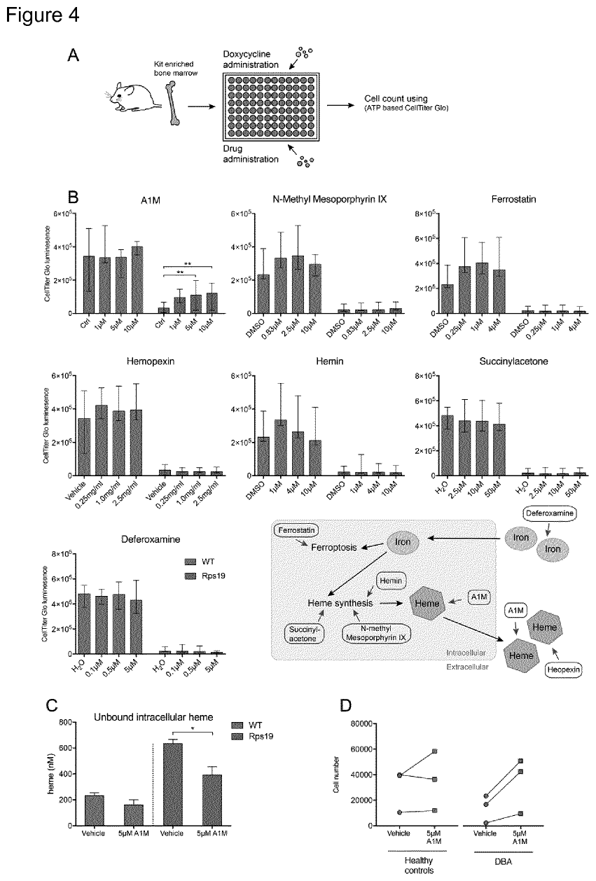

The effects of radiation on the bone marrow and peripheral blood cells were evaluated 4 days after injection of 150 MBq ¹⁷⁷Lu-DOTATATE using flow cytometry and Sysmex hematological analyzer. It was observed that the percentage of viable reticulocytes was significantly reduced in both bone marrow and peripheral blood following exposure to ¹⁷⁷Lu-DOTATATE (FIG. 2). Subcutaneous co-administration of A1M, deposited 30 minutes prior to the ¹⁷⁷Lu-DOTATATE administration, maintained a completely preserved reticulocyte population at the level of the non-radiation exposed control animals (FIG. 2).

### Effects of Radiation on Terminal Erythroid Differentiation

The terminal erythroid differentiation within the bone marrow was evaluated 4 days after the injection of 150 MBq ¹⁷⁷Lu-DOTATATE. In addition to the effects seen on reticulocytes (in line with that of FIG. 2) a clear effect, although not statistically significant, was also seen on the proerythroblast population (FIG. 3, population denoted I). No effect of radiation was observed in any of the other progenitor populations (denoted population II-IV). Subcutaneous co-administration of A1M, deposited 30 minutes prior to the ¹⁷⁷Lu-DOTATATE administration, displayed protection of the proerythroblast population, in addition to the reticulocytes, and maintained them at the level of the non-radiation exposed control animals (FIG. 3).

### Example 2—A1M Reduces Excess Intracellular Heme and Improves Proliferation in Diamond-Blackfan Anemia

In this study, we show that human recombinant A1M (A1M) is the only compound tested which lead to an increased proliferation of murine Rps19 deficient erythroid precursors along with an ability to reduce the level of unbound heme.

### INTRODUCTION

Diamond-Blackfan anemia (DBA) is a congenital disorder where patients show macrocytic anemia and a scarcity of erythroid precursors in the bone marrow. Around ˜70% of all patients have mutations in ribosomal proteins, most commonly in RPS19. Protein translation in general and translation of certain mRNA in particular are altered in DBA, contributing to the disease phenotype. The tumor suppressor p53 is hyperactivated in DBA, resulting in decreased proliferation and increased apoptosis in erythroid precursors. Current treatments for DBA are glucocorticoids, blood transfusions, or allogenic bone marrow transplantation. Unfortunately, all available therapies have side effects impairing the quality of life for the patients. For this reason, there is an urgent need for disease specific treatments for DBA.

It has already been demonstrated that erythroid precursor cells from a DBA patient contain pathologically high intracellular heme levels, which could explain poor erythroid cell proliferation. Since unbound heme is toxic, the increase in intracellular heme needs to be met by equivalent amounts of globin to generate hemoglobin, the essential oxygen carrying molecule of red blood cells. In DBA however translation is impaired and in erythroid cells the main synthesized proteins are globins. This finding suggest that drugs reducing heme toxicity are potential treatment strategies for DBA, by either enhancing globin mRNA translation, which is the rationale behind an ongoing clinical trial with Leucin, or by reducing intracellular heme levels.

In this example, we screened for novel therapeutic strategies for reducing toxic unbound intracellular heme in DBA. While drugs inhibiting heme synthesis failed to improve proliferation of Rps19 deficient erythroid progenitor cells, treatment with the heme scavenger A1M resulted in reduction of elevated intracellular heme levels and increased proliferation of erythroid cells from both a murine model for DBA, and DBA patients.

### Methods

### Drug Treatment of Murine Bone Marrow Cells

Ethical permission was granted by a local ethical committee for all animal research. Bone marrow from inducible Rps19 deficient mice of 8-14 weeks was enriched for CD117 (Kit) expression using magnetic beads (Miltenyi, Germany) according to manufacturer's instructions. Cells were cultured in StemSpan serum free expansion medium (SFEM) (Stem cell technologies, Canada), 1% penicillin/streptomycin (GE Healthcare, US), 10% fetal bovine serum (ThermoFisher Scientific, US), 100 ng/ml mSCF (Peprotech, US), 300 μg/ml h-holo-transferrin (Sigma-Aldrich, US), and 2 U/ml hEpo (Johnson-Johnson, US) with 0.2 μg/ml Doxycycline (Sigma-Aldrich).

The drugs administered 24 hours after Doxycycline administration were A1M (supplied by A1M Pharma AB, Sweden), N-methyl mesoporphyrin IX (AH Diagnostics, Denmark), Succinylacetone, hemin, Deferoxamine, Ferrostatin and N-acetyl-L-cystein and hemopexin (Sigma-Aldrich). Cell expansion 5 days after drug administration was measured using CellTiter Glo (Promega, US), which measures the number of viable and metabolically active cells in culture based on quantitation of the ATP present. Plates were read on Victor 3 Multilabel counter (PerkinElmer, US).

### A1M Treatment of Human Samples

Peripheral blood samples were collected at Lund University hospital using informed consent according to ethical permission granted by the Swedish ethical review board. The three DBA patients had mutations in RPS19, RPS26 and RPL35a respectively, and all received blood transfusions with chelation therapy. Mononuclear cells from DBA patients and healthy subjects were obtained using lymphoprep (Fresenius Kabi, Germany) and enriched for CD34 expression using magnetic beads (Miltenyi, Germany) according to manufacturer's instructions. Cells were cultured in SFEM (Stem cell technologies), 1% penicillin/streptomycin (GE Healthcare), 100 ng/ml hSCF (Peprotech), 2 U/ml Epo (Johnson-Johnson). 5 μM A1M or vehicle (Tris-HCl/NaCl) were added every 2-3 days for a total of 7-8 days. Cell count was performed manually using a hemocytometer.

### Results and Discussion

### A1M Increases Proliferation of Erythroid Rps19 Deficient Cells

Since increased intracellular heme levels may contribute to impaired erythroid precursor proliferation in DBA patients, we performed a drug screen for compounds affecting iron or heme availability in erythroid cells from a DBA mouse model (FIG. 4A). None of the compounds affecting heme synthesis or iron availability improved proliferation of Rps19 deficient cells. Strikingly, A1M (at 5 and 10 μM) was the only compound tested showing increased proliferation of Rps19 deficient erythroid precursors. No effect on proliferation was seen in WT cells, indicating a specific effect of A1M only in Rps19 deficient erythroid precursors (FIG. 4B).

### A1M Lowers Elevated Heme Levels in Rps19 Deficient Cells

A1M protects against heme induced cell and tissue damage by scavenging and degrading heme. In Rps19 deficient erythroid cells A1M was shown to significantly reduce the level of unbound intracellular heme back to WT levels (FIG. 4C). Since treatment with the mainly extracellular heme scavenger hemopexin had no effect on DBA cells (FIG. 4B), A1M likely functions intracellularly on erythroid DBA cells.

Early Erythroid Cells from DBA Patients Increase Proliferation at A1M Treatment

Purified erythroid precursors from three DBA patients cultured with 5 μM A1M all showed improved expansion, while no such trend was observed in cells from healthy subjects (FIG. 4D).

In summary, this study identifies the heme binding protein A1M to increase proliferation in erythroid cells from DBA patients, by normalizing the levels of unbound intracellular heme. Our findings suggest that A1M has the potential to reduce heme toxicity in anemic conditions caused by ribosomal protein deficiency, such as del 5q-myelodysplastic syndrome. Taken together, this study has identified that A1M can be used to treat cells from DBA patients. It also serves as a proof of concept study that targeting heme levels could be used in developing more disease specific DBA therapies.

### Example 3—Evaluation of Hematopoietic Recovery after Several Different Inducers of Bone Marrow Damage

Study the effect of A1M treatment on bone marrow recovery after a number of different damage, including exposure to whole body irradiation, genotoxic and cytotoxic molecules (such as 5-FU, cisplatin etc.), hemolysis induced by agents such as Phenylhydrazine or damage caused by genetic defects such as RPS19-deficiency in Diamond-Blackfan anemia.

The above will be evaluated by the following means:

### 1. Serial Transplantations in Mice for Evaluating Stem/Progenitor Recovery.

Gold standard experiment is to perform serial transplantations as well as limited-dilution experiments (Frisch et al. 2014, Rundberg Nilsson et al. 2015). Serial transplantations means that bone marrow from damaged mice (see the different damage above) that were A1M treated or non-treated will be re-transplanted to irradiated mice together with competitor cells at least twice. This is to demonstrate that long-term stem cells are preserved in A1M treated mice.

### 2. Limited-Dilution Experiments in Mice.

Limited-dilution experiments are used to functionally quantify stem cells (Bonnefoix et al. 2010). Different numbers of bone marrow cells from A1M treated and non-treated mice will be re-transplanted to irradiated mice together with healthy competitor cells. Based on the level of hematopoietic reconstitution from treated mice compared to healthy cells it is possible to estimate the number of stem cells that survived the damage.

3. FACS and Colony Assays after Treatment in Mice.

Standard experiments to be performed to evaluate number of progenitor cells. FACS analysis will determine if A1M therapy leads to increased survival of hematopoietic progenitor cells and stem cells.

### 4. A1M Uptake in Stem/Progenitor Cells.

A1M will be injected into healthy and damaged (see above) mice. FACS will then be used to sort stem and progenitor cells from the animals and determine the uptake of A1M.

### 5. A1M Knockout Mice.

The above experiments are performed in animals with normal endogenous levels of A1M and are performed to evaluate the potential of A1M as a drug. To more clearly determine mechanism of A1M in protecting hematopoietic stem/progenitor cells during bone marrow damage the above experiments will be performed on A1M knockout mice that are deficient of A1M.

### REFERENCES

Bonnefoix, T. and M. Callanan (2010). “Accurate hematopoietic stem cell frequency estimates by fitting multicell Poisson models substituting to the single-hit Poisson model in limiting dilution transplantation assays.” Blood 116(14): 2472-2475.
Frisch, B. J. and L. M. Calvi (2014). “Hematopoietic stem cell cultures and assays.” Methods Mol Biol 1130: 315-324.
Rundberg Nilsson, A., C. J. Pronk and D. Bryder (2015). “Probing hematopoietic stem cell function using serial transplantation: Seeding characteristics and the impact of stem cell purification.” Exp Hematol 43(9): 812-817 e811.

1. Alpha-1-microglobulin (A1M) for use in the protection of bone marrow cells in a subject.

2. A1M for use in the protection of one or more of hematopoietic stem cells and progenitor cells residing in the bone marrow or other hematological niches of a subject.

3. A1M for use according to claim 1 or 2, wherein the protection is for damage caused by exposure to ionizing radiation, chemotherapy or genotoxic substances.

4. A1M for use according to claim 3, wherein the radiation is ionizing radiation, which emanates from a source external to the body such as in external beam radiotherapy or X-ray radiography.

5. A1M for use according to claim 3, wherein the radiation is ionizing radiation, which emanates from a source internalized in the body such as unsealed source radionuclide therapy (RNT); peptide receptor nuclide radiation therapy (PRRT); radioimmunotherapy (RIT) or brachytherapy.

6. A1M for use according to any of the preceding claims, wherein the protection is for ionizing radiation used for diagnostic purposes.

7. A1M for use according to claim 5, wherein a compound labelled with radionuclide is selected from the group consisting of receptor ligands, Affibody molecules, Diabodies, antibody fragments, and/or other small molecules.

8. A1M for use according to claims 5-7, wherein a somatostatin analogue is labelled with radionuclide.

9. A1M for use according to claim 8, wherein the somatostatin analogue is selected from the group consisting of octreotide, lanreotide, Tyr³-octrotide, Tyr³-octrotate, DOTADOC, DODATATE, DOTA-lanreotide, pasireotide, dopastatin and octreotide LAR.

10. A1M for use according to any of claims 4-9, wherein A1M is administered before, at the same time or after exposure to radiation.

11. A1M for use according to claim 10, wherein A1M is infused over a period of time.

12. A1M for use according to any of claims 4-11, wherein radiation and administration of A1M are essentially simultaneous.

13. A1M for use according to any of claims 4-11, wherein A1M is administered on more than one occasion before or after radiation.

14. A1M for use in the treatment of bone marrow cell damages.

15. A1M for use in the treatment of one or more of hematopoietic stem cells and progenitor cells residing in the bone marrow and other hematological niches.

16. A1M for use according to any of claims 3-15, wherein the damages result in a decreased production of one or more of hematopoietic stem cells and progenitor cells, such as but not limited to, reticulocytes, compared with a control or a sample taken from the same subject before any exposure to radiation, chemotherapy or genotoxic substances.


SEQUENCE LISTING (17 sequences)

SEQ ID NO: 1 - protein, 183 aa, Homo sapiens
  Wildtype human A1M, no mutations
GPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 2 - protein, 184 aa, Homo sapiens
  rhA1M, i.e. N-terminal Met
MGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 3 - DNA, 549 bp, Homo sapiens
  Wildtype human A1M, no mutations
ggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

SEQ ID NO: 4 - DNA, 603 bp, Homo sapiens
  rhA1M, i.e. N-terminal Met
atgcatcaccatcaccatcaccatcacggtggaggagggggtatcgagggccgcggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaggtttctctatcacaaatccaaatggaacataaccatggagtcctatgtggtccacaccacctatgatgagtatgccatttttctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgaga

SEQ ID NO: 5 - protein, 184 aa, Homo sapiens
  rhA1M, No tag, N-terminal met, N17,96D, R66H
MGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 6 - protein, 184 aa, Homo sapiens
  hA1M, not tag, N-terminal Met, M41K
MGPVPTPPDNIQVQENFNISRIYGKWYNLAIGSTCPWLKKIKDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 7 - protein, 195 aa, Homo sapiens
  6His, N17,96D; R66H
MHHHHHHDDDDKGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 8 - protein, 195 aa, Homo sapiens
  hA1M, 6His, M41K
MHHHHHHDDDDKGPVPTPPDNIQVQENFNISRIYGKWYNLAIGSTCPWLKKIKDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 9 - protein, 197 aa, Homo sapiens
  8His, N17,96D; R66H
MHHHHHHHHDDDDKGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 10 - protein, 197 aa, Homo sapiens
  hA1M, 8His, M41K
MHHHHHHHHDDDDKGPVPTPPDNIQVQENFNISRIYGKWYNLAIGSTCPWLKKIKDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 11 - protein, 197 aa, Homo sapiens
  hA1M, 8His, no mut
MHHHHHHHHDDDDKGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 12 - protein, 180 aa, Homo sapiens
  hA1M, No tag, N-terminal Met, N17,96D; R66H; truncated
MGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPI

SEQ ID NO: 13 - protein, 184 aa, Homo sapiens; identical to SEQ ID NO: 6
  hA1M, not tag, N-terminal Met, M41K; truncated

SEQ ID NO: 14 - protein, 191 aa, Homo sapiens
  6His, N17,96D; R66H; truncated
MHHHHHHDDDDKGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPI

SEQ ID NO: 15 - protein, 195 aa, Homo sapiens; identical to SEQ ID NO: 8
  hA1M, 6His, M41K; truncated

SEQ ID NO: 16 - protein, 193 aa, Homo sapiens
  8His, N17,96D; R66H; truncated
MHHHHHHHHDDDDKGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPI

SEQ ID NO: 17 - protein, 197 aa, Homo sapiens; identical to SEQ ID NO: 10
  hA1M, 8His, M41K; truncated